(19) United States
(12) Patent Application Publication
(10) Pub. No.: US 2006/0064151 A1
(43) Pub. Date: Mar. 23, 2006

(54) Cranial aneurysm treatment arrangement

(76) Inventors: Lee R. Guterman, Amherst, NY (US); Elad I. Levy, Amherst, NY (US)

Correspondence Address: Donald N. Halgren, 35 Central Street, Manchester, MA, 01944, US

(21) Appl. No.: 11/232,393

(22) Filed: Sep. 21, 2005

Related U.S. Application Data
(60) Provisional application No. 60/612,128, filed on Sep. 22, 2004.

(51) Int. Cl. A61F 2/06

(52) U.S. Cl. 623/1.3

(57) ABSTRACT

A stent for application within an aneurysm the stent comprising an elongated tubular member having a proximal end and a distal end. The stent has a proximal portion expandable from a first diameter to a second diameter. The distal end of the stent may be expandable to a third diameter, which third diameter is larger than the second diameter. The stent may be left disposed across the neck of the aneurysm.

### BACKGROUND OF THE INVENTION

1. Field of the Invention

This invention relates to brain aneurysms and more particularly to apparatus and methods of using such apparatus for treating such brain aneurysms, based upon Provisional Patent application 60/612,128, filed Sep. 22, 2004, and incorporated herein by reference in its entirety.

2. Prior Art

An aneurysm is an abnormal increase in the shape or diameter of a blood vessel. Such a condition is typically treated with a micro catheter to deliver coils to the site of the enlarged or misshapen blood vessel which has been occluded by the aneurysm from within the blood vessel. A basilar tip aneurysm is a particular aneurysm that occurs at the distal bifurcation of the basilar artery, between the origin of the two posterior cerebral arteries or branches. Current endovascular treatments fill the aneurysms with various materials in an attempt to limit the blood flow into those aneurysms from the native blood circulation. Platinum coils or liquid embolic agents may be delivered into the aneurysm by a micro catheter. Such delivery however, may result in that material being discharged into the native blood circulation.

It is an object of the present invention to overcome the disadvantages of the prior art.

It is a further object of the present invention, to provide an apparatus and associated methodology with that apparatus to treat any intracranial aneurysms that occur, and specifically at vascular bifurcations of the brain.

It is a further object of the present invention, to provide an apparatus and methodology in a further preferred embodiment thereof, which may be utilized in any aneurysm in a body.

### BRIEF SUMMARY OF THE INVENTION

The present invention relates to a device and the method of using that device for endovascular occlusion of a damaged blood vessel, for example, a basilar aneurysm.

Such a device comprises an adaptable stent for treatment of aneurysms at bifurcations within the vasculature. A stent is a cylindrical device preferably made from nitinol or a similar shape memory material. Such a stent device may also be woven of similar or dissimilar wires or fabric of similar or dissimilar diameter. Such stent devices may also be made from hypotubes. Stents utilized in cranial vasculature may preferably be delivered through a micro catheter to the bifurcation being treated. The diameter of such stents for such treatment may range from about 0.010 inches to about 5.0 inches, depending of course upon its intended ultimate location of placement in the body. It is delivered to the aneurysm and may be positionally adjusted and retrieved, if necessary, through the micro catheter, by a push wire. Portions of the stent device may be made from and/or coated with a material woven from marker bands such as platinum, gold and/or Nitinol or other metals known in the treatment art which provide high opacity to permit visualization of that stent during placement thereof. The delivery tube itself may also, in a further preferred embodiment, be made from, or coated with, for example, a material such as gold or platinum for improved opacification and visualization of the stent implantation procedure.

In the delivery of such a stent device into an aneurysm located on the site of a bifurcation, the distal tip of the delivery catheter is first positioned into the aneurysm itself so that the distal tip of the catheter is within central portion of the aneurysm. The stent device is then pushed by its push wire, out the distal end of the catheter and is permitted to expand both within the aneurysm itself and also to span the neck of the aneurysm, so as to also be placed within the parent vessel, which parent vessel was utilized as a delivery path for the stent device.

The stent device, as aforesaid, preferably has at least portions thereof comprised of a self expanding material such as nitinol.

In one preferred embodiment of the present invention, the stent has a narrowed waist portion which is to be disposed just outwardly of the opening of the aneurysm between several branches of the parent vessel. The proximal end of the stent device is arranged to anchor the stent upon expansion thereof, within the parent vessel and thus anchor the distal end of the stent device, within the aneurysm itself.

Thus, a somewhat hemispherical configuration of the distalmost end of the stent device becomes fixed within the aneurysm, and a narrowed or pinched portion of the stent device becomes situated at the juncture of the branches and the parent vessel. The stent is thus positioned to span the neck of the aneurysm. Blood flow is thus permitted through the parent branches, and the aneurysm itself becomes blocked by the generally hemispherical-shaped distal portion of the stent device.

Further preferred embodiments of the present invention may be comprised of an elongated stent device having a cone-shaped or enlarged distalmost end, flared out due to modification of the weave of material thereat, or due to self-straightening or expandable strut portions extending diametrically across the distalmost end, or within the cone shaped portion adjacent the distal end of the stent device, or woven within the material comprising the weave at the distal most end of the stent device.

In another further preferred embodiment of the present invention, the stent may have a distal end, which is received within the aneurysm, the central body portion and the proximal end thereof extending across the neck of the aneurysm and into the parent vessel. The distal end of the stent in this embodiment being non-expandable. Such distal end portion may preferably be woven or cut from a different material that the remainder of the stent body.

The present invention includes a further embodiment wherein a mesh device formed of a flexible mesh material of wire formed plastic may be conformed so as to be longitudinally advanceable through a micro-catheter by a push shaft so as to deform the diameter thereof sufficiently so to pass slightly within the orifice and neck portion of an aneurysm between a pair of branch vessels extending from a parent vessel. The mesh device of this embodiment has a rounded distalmost end and a generally cylindrical body. Such a mesh stent-like device may be preferably of thimble-shape, having its distal end hemispherically-shaped so as to easily enter the orifice of the aneurysm. The mesh portion of the body is positioned between the branches extending from the parent vessel. The distal end of the mesh implant device covers the orifice. An eternally disposed embolitic material delivery catheter may be longitudinally advanceable and retractable within the micro-catheter, so as to deliver embolitic material within the aneurysm itself. The mesh stent-like device may have a slightly tapered proximal body portion in a further embodiment thereof, so as to permit an externally displaceable embolitic material delivery catheter to be disposed between the exterior of the micro-catheter and the wall of the parent vessel, thus permitting embolitic material to be delivered to the aneurysm just outside of the body portion of the mesh stent device.

A yet further embodiment of the present invention comprises an elongated mesh sleeve having a proximal open end and a tapered distal closed or leading edge end. The mesh sleeve may be made in one preferred embodiment, of a shape memory material, such as a shape memory metal alloy, or a shape memory thermoplastic. The internal control shaft or push rod is preferably utilized to deliver the tapered-end sleeve through a micro-catheter into the central portion of an aneurysm disposed at a bifurcation. The distal portion of the tapered sleeve through its shape memory means, or through its attachment to the control shaft, may be folded proximally so that the tapered otherwise distal-edge, is now pulled or folded back within the main body portion of the sleeve. Thus, a cup-shaped mesh, now comprising a double thickness, sits within the lower girth of the aneurysm at the bifurcated orifice. The control shaft may be removed from the tapered tip of the leading edge of the sleeve by a release joint or by electrolytic action or a dissolvable connection there-between. Flow of blood is preferably through the center of the device and out to the side branches to minimize flow into the aneurysm, but in further embodiments, the diameter of the main body portion of the mesh sleeve may be such as to permit blood flow around the circumference thereof, as well as the major blood flow through the openings within the mesh. Utilizing an inverted form of the tapered sleeve permits a mesh material of relatively wider openings therethrough, because by virtue of the doubling back and folding over of the mesh material during its inversion effectively one thus creates a mesh of comparable porosity (two layers) to that of an otherwise tighter mesh, so as to comparatively inhibit embolitic material from escaping the aneurysm once that device has been put in place.

A further embodiment of the present invention comprises a mesh implant having a rather bulbous distal- or leading end, and a generally cylindrical body portion comprising its proximal end. The proximal end is open to permit the use of a pushrod or control rod, allowing the expandable bulbous end to be narrowed during its deployment within aneurysm between a pair of side branches. Upon release of the control rod from the distal end of the device, the bulbous portion assumes its enlarged configuration with a slightly tapering proximal portion of the bulbous end nesting within the orifice defining the aneurysm. The tapered, narrowed-diameter portion of the body of the present device is foraminous end of sufficient narrow diameter, so as to permit blood flow therearound and therethrough, while the bulbous portion prevents embolitic material from escaping into the side branches.

The bulbous device of the aforementioned embodiment may include an arrangement of struts or shape memory members of metal or thermoplastic, formed across the proximal portion of the bulbous end of the stent device. The device is positioned at a bifurcation with its bulbous end within the aneurysm. A staging, or cross cut strut arrangement, is positioned preferably at the orifice of the aneurysm. A micro-catheter may be positioned through or adjacent the proximal body portion of the device to deliver embolic material into the aneurysm. The stage, or cross struts, help to contain the embolitic material within the aneurysm, and prevent it from passing the transition point between the bulbous portion and the tubular body portion of the device. The embolitic material will be utilized, of course, to fill the bulbous end and support its treatment. The struts or stage in preferential alignment with the orifice of the aneurysm acts as a partial wall between the branches of the bifurcation.

A further embodiment of the bulbous cranial aneurysm treatment arrangement of the earlier embodiments, is contemplated with a bulbous mesh distalmost end having a generally mid-portion pinched to a neck by a constriction band. The constriction band separates the bulbous and distal end from the tubular end of the implant. The tubular end of the implant may be somewhat more porous, having larger openings therethrough or selectively having positioned holes circumferentially about the tubular end, immediately adjacent the constriction band. Those large holes would permit flow of blood through, across, and between the various branches subtending the parent vessel. The bulbous distal end of the device may be filled with a liquid or solid embolitic material, which has been delivered by a micro-delivery catheter through the center of the tubular device and the constriction band, or alongside the tubular portion and directly into the distalmost bulbous portion of the device. The hose positioned adjacent the constriction band, permits blood flow therethrough, as well as the narrowed constricted portion providing blood flow around that segment of the device.

A further embodiment comprises an elongated micro-catheter-delivered aneurysm treatment device having a bulbous distal or leading end, and a tubular proximal, or trailing end, separated by a pinched waist portion through which a micro-catheter delivery device is pushed. The micro-catheter delivery device has its distal end within the bulbous portion of the treatment device, with its pinched or transitioned waist portion best disposed just at the neck of the aneurysm, so as to minimize any potential blockage between the side branches of the parent vessel. The micro-catheter delivery device, temporarily disposed within the bulbous portion of the treatment device, is utilized to deliver, for instance, an embolic coil or other embolic material within the aneurysm itself, the pinched or waist portion, minimizing any migration ever of embolic material of the aneurysm itself.

Further embodiments of the aneurysm treatment devices consist of mesh-like sleeves passing either open distalmost ends or bulbous closed distalmost ends and open proximal ends. Each of these devices is characterized with a plurality of side holes arranged through the wall portions of the mesh device, so as to maximize flow of blood between adjacent side branches of the parent vessel. The pinched, or tapered portions of the treatment device minimizes and/or restricts the embolitic material from moving out of the aneurysm itself. Further embodiments of that same concept involve the use of struts or stages immediately adjacent the side holes within the wall of the tubular portion of the treatment device.

A still yet further embodiment of the present invention includes an elongated aneurysm treatment device, having an open proximal body portion and a cup-shaped distalmost portion, between which lies a pinch waist segment. A micro-catheter extends through the entire treatment device having a distal end extending beyond the distal end of the treatment device and centrally within the aneurysm itself. Embolic material is thus delivered within the aneurysm and will be constrained therewithin. The pinched or waist portion, or possibly a constriction band, is positioned at the bifurcation, so as to permit blood flow therearound. The distalmost end of the micro-delivery catheter extends within the aneurysm itself, and extends distally beyond the distalmost end of the cup-shaped treatment device, disposed within the neck of the aneurysm.

Thus there has been shown a unique stent configuration and delivery arrangement for the treatment of intracranial aneurysms that occur at bifurcations of the brain such as, for example, the basilar apex, the middle cerebral artery, the anterior communicating artery, or the internal carotid bifurcation. Such treatment may in further preferred embodiments be utilized for aneurysms located anywhere in the body.

### BRIEF DESCRIPTION OF THE DRAWINGS

The objects and advantages of the present invention will become more apparent when viewed in conjunction with the following drawings in which;

FIG. 1 is a side elevation view of a bifurcated vessel with an aneurysm therein, and a stent device placed within that aneurysm;

FIG. 2 is a further embodiment of the stent device of the present invention;

FIG. 3A is a yet a further embodiment of a stent device of the present invention;

FIG. 3B shows a stent device with an arrangement of struts/stage placed within a mid-portion of the stent, the struts located within a neck portion of an aneurysm;

FIG. 3C shows a stent device with an arrangement of struts/stage at a distal portion of the stent device, those struts located within a neck portion of an aneurysm.

FIG. 4 is yet still a further embodiment of the present invention showing a stent placed across the neck of an aneurysm, with the distal end of the stent in an unexpanded configuration;

FIG. 5A is a side elevational view of an aneurysm at a bifurcation, showing a thimble-shaped treatment device disposed therewithin;

FIG. 5B is a side-elevational view of the thimble-shaped treatment device shown in FIG. 5A;

FIG. 6A is an elongated sleeve-shaped treatment device having a tapered distal tip thereon;

FIG. 6B shows the treatment device of FIG. 6A in an inverted configuration, forming a somewhat hemispherically shaped mesh device;

FIG. 7A is a side elevational view of a bulbous treatment device of the present invention;

FIG. 7B is a side elevational view of the bulbous treatment device shown in FIG. 7A, disposed within an aneurysm;

FIG. 8A is a side elevational view of a bulbous treatment device in a further embodiment thereof, showing cross struts at the neck of the device;

FIG. 8B is the treatment device shown in FIG. 8A arranged within a bifurcated aneurysm, with the placement of the struts across the neck of the device and also generally across the neck of the aneurysm;

FIG. 9A is a side elevational view of an elongated aneurysm treatment device with a constriction band shown therewith;

FIG. 9B is a side elevational view of the treatment device shown previously in FIG. 9A arranged within a bifurcated aneurysm;

FIGS. 10A-C show side elevational views of an aneurysm treatment device with a microcatheter delivery device arranged therewith;

FIGS. 11A-D show side elevational views of aneurysm treatment devices with side holes thereon;

FIGS. 12A-B show side elevational views of various inventive aneurysm treatment devices in a further embodiment thereof; and

FIG. 13 is a side elevational view of a further aneurysm treatment device with a micro-delivery catheter disposed therethrough, all shown arranged within a bifurcated vessel having an aneurysm thereon.

### DETAILED DESCRIPTION OF THE PREFERRED EMBODIMENTS

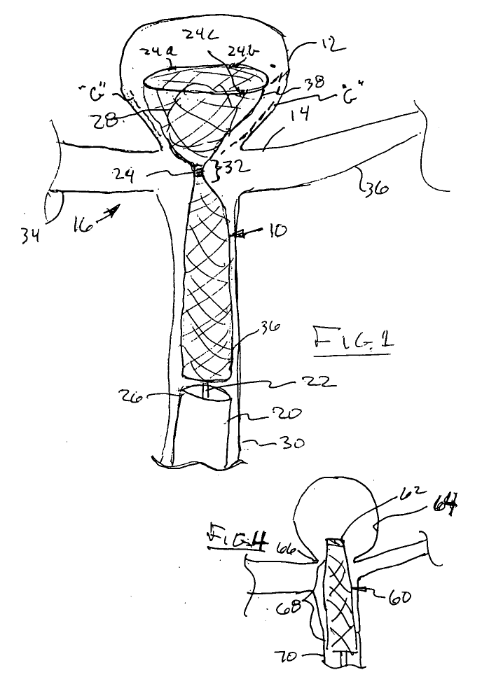

Referring now to the drawings in detail, and particularly to FIG. 1, there is shown the present invention which comprises an adaptable stent device 10 for treatment of an aneurysm 12 located at a bifurcation 14 within the cranial vasculature 16. The first preferred embodiment of the stent 10 is a cylindrical device made from nitinol or a similar shape memory material. Such a stent device may also be woven of similar or dissimilar wire or fiber/fabric material of similar or dissimilar diameter. Such stent devices may also be made from laser cut hypotubes. Stents utilized in cranial vasculature may preferably be delivered through a micro catheter 20 to the bifurcation 14 being treated. The diameter of such stent devices 10 for such treatment may range from about 0.010 inches to about 0.050 inches. The stent device 10 may be delivered to, be repositioned and/or withdrawn from the aneurysm 12 through the micro catheter 20, by a thin push wire 22, which extends through the delivery catheter 20, as represented in FIG. 1. Annular portions of the stent device 10 as well as all of and/or portions of the delivery catheter 20 may be formed of, coated with and/or woven from marker bands 24 (as well as peripheral marker bands 24a, 24b & 24c, on the distal periphery of the stent, as shown in FIG. 1), such as platinum or gold alone or in combinations thereof, which provide high opacity to permit visualization of that stent device 10 and delivery catheter 20 during placement of such stent device 10 into and across an aneurysm. Such a neck-located marker band 24 may also comprise a stent-device 10 neck-constrictor.

In the delivery of such a stent device 10 into an aneurysm 12 located on the site of a bifurcation 14, the distal tip 26 of the delivery catheter 20 is first positioned into the aneurysm 12 itself so that the distal tip 26 of the catheter 20 is within central portion of the aneurysm 12. The stent device 10 is then pushed by its push wire 22, out the distal end 26 of the catheter 20 and is thus permitted to expand (by self expansion in one embodiment or by a controllably expandable micro balloon 28 in a further embodiment, which balloon 28 in yet a further embodiment may be dissolvable) both within the aneurysm 12 itself and also expand within the parent vessel 30. Such expansion within the parent vessel 30 provides anchoring of the stent 10 within the vasculature 16. The parent vessel 30 is utilized as a delivery path for the stent device 10. The stent device 10 is preferably disposed across the neck of the aneurysm 12, as shown for example, in FIG. 1.

The stent device, as aforesaid, has at least portions thereof comprised of a self expanding material such as nitinol.

In one preferred embodiment of the present invention, the stent has a narrowed waist portion 32, as shown in FIG. 1 which is to be disposed just outwardly of the opening of the aneurysm between several branches 34 and 36 of the parent vessel 30. The proximal end 36 of the stent device 10 is arranged to anchor the stent, upon expansion thereof, within the parent vessel 30 and thus anchor the distal end 38 of the stent device 10 within the aneurysm 12 itself.

Thus, preferably a somewhat hemispherical configuration of the distalmost end 38 of the stent device 10 becomes fixed within the aneurysm 12 and a narrowed or pinched portion 32 of the stent device 10 becomes situated at the juncture of the branches 34 and 36 and the parent vessel 30. Blood flow is thus permitted around the waist 32 of the stent device 10 and through the parent vessel 30 and branches 34 and 36. The aneurysm 12 itself becomes blocked by the generally hemispherically shaped distal portion 38 of the stent device fitting within and preferably against the inner side of the aneurysm 12, as represented by dashed lines “G” in FIG. 1.

Further preferred embodiments of the present invention may for example be comprised of an elongated stent device 40, having a cone shaped or enlarged distalmost end 42, as represented in FIG. 2. The distal end 42 may be flared out due to modification or a co-weave of different materials incorporated within the weave of material 44 thereat, or due to self straightening or expandable strut members 46 extending diametrically across the distalmost end 42.

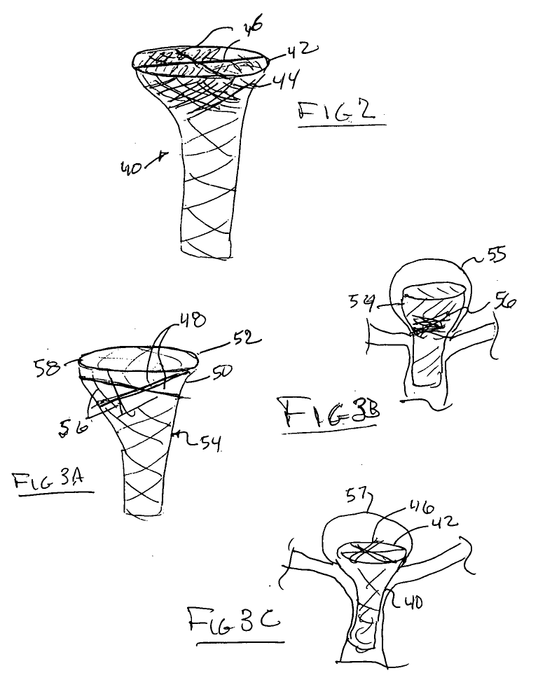

Alternatively, self-expandable struts 48 (for example, nitinol material) may be disposed within or across the cone shaped portion 50 adjacent the rim 52 of the stent device 54, as shown in FIG. 3A or alternatively, an arrangement of expandable struts 56 may be woven as wires within the material comprising the weave at the distalmost 58 end of the stent device 54, as represented in FIG. 2. Those struts 56, in a further embodiment may delivered by a further micro-delivery catheter and released within the stent 40 subsequent to its insertion within an aneurysm. Such struts 56 are utilized to hold embolic material within the stent 40/54 when such stent is placed within an aneurysm. FIG. 3B represents the distally-‘adjacent” struts 56 when the stent 54 is disposed within an aneurysm 55, and FIG. 3C represents the arrangement of struts 46 actually at the distal end 42 of the stent 40, across the neck of an aneurysm 57.

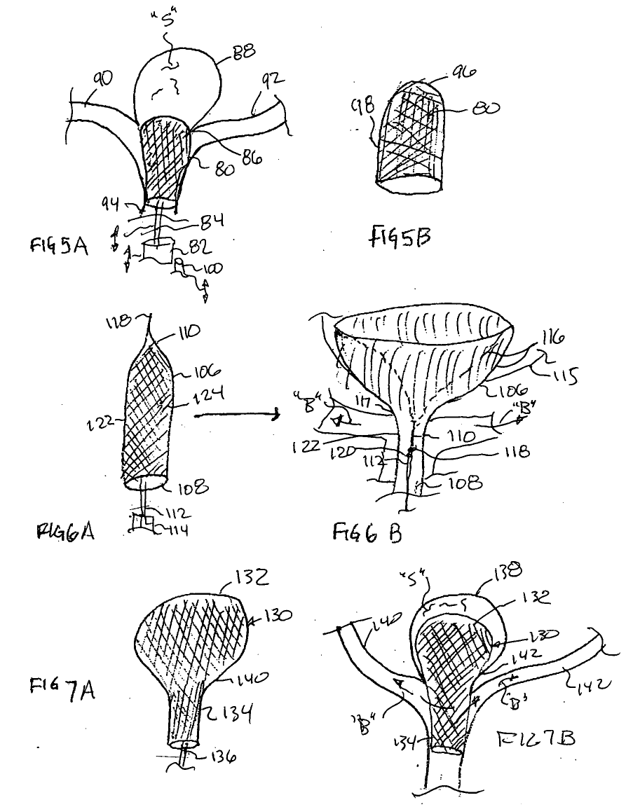

The stent device 60 is shown in FIG. 4 having its distal end 62 thereof placed within an aneurysm 64. Such distal end 62 being made of or woven from a dissimilar material in this embodiment which is non or unexpandable. The stent device 60 having a body portion 68 which extends across the neck 66 of the aneurysm 64 and into the parent vessel 70. The body portion 68 is preferably self expandable or it may be expended by a micro-balloon, not shown for clarity.

The present invention also includes a further embodiment, represented in FIGS. 5A and 5B, wherein an elongated, generally tubular mesh device 80 may be formed of, for example, a flexible, shape-memory foraminous material of machined metal, woven wire or molded, machined or otherwise thermo-formed plastic that may be conformed so as to be longitudinally stretchable and thus of reduceable diameter to permit advancement through a micro-catheter 82 by a manually controllable push shaft 84 so as to deform the diameter thereof sufficiently so to pass slightly within the orifice and neck portion 86 of an aneurysm 88 between a pair of branch vessels 90 and 92 extending from a parent vessel 94. The mesh device 80 of this embodiment has a rounded distalmost end 96 and a generally cylindrical body 98. Such a mesh stent-like device 80, preferably of thimble-shape, having its distal end 96 hemispherically-shaped so as to easily enter/press against the orifice 86 of the aneurysm 88. The mesh body portion 98 of the device 80 is positioned between the side branches 90 and 92 extending from the parent vessel 94. The distal end 96 of the mesh implant device 80 covers the orifice 86 of the aneurysm 88. An externally disposed embolic/embolitic material delivery catheter means 100 may be longitudinally advanceable and retractable within the micro-catheter 82, or a stent-adjacent micro-catheter, so as to deliver embolic material “S” within the aneurysm 88 itself. The mesh-like stent device 80 may have its body portion 98 slightly tapered in a further embodiment thereof, so as to permit the externally displaceable embolitic material delivery catheter 100 to be disposed between the exterior of the micro-catheter 82 and the wall of the parent vessel 94, thus permitting the embolitic material “S” to be delivered to the aneurysm 88 by movement just outside of the body portion 98 of the mesh stent device 80. By increasing the mesh density of the stent device represented for example, in FIGS. 5a and 5B, such a stent may be utilized to permanently occlude any vessel.

A yet further embodiment of the present invention represented in FIGS. 6A and 6B comprises an elongated mesh sleeve 106 having a proximal open end 108 and a tapered distal closed or leading edge end 10. The mesh sleeve 106 may be made in one preferred embodiment, of a shape memory material, such as a shape memory metal alloy for example, Nitinol, or a shape memory thermoplastic. An internal control shaft or push rod 112 is preferably utilized to deliver the tapered-end sleeve 106 through a micro-catheter 114 into the central portion of an aneurysm 115, disposed at a bifurcation. The distal portion 110 of the tapered sleeve 106, through a shape memory means, or through its attachment to the control shaft 112, may be pulled/folded proximally, as represented in FIG. 6B, so that the tapered otherwise distal-edge portion 110, is now pulled or folded back within the main body portion of the sleeve 106. Thus, a cup-shaped mesh, now comprising a double thickness 116, sits within the lower girth of the aneurysm 115 at the bifurcated aneurysm orifice 117. The control shaft 112 may be removed from the tapered tip 118 of the leading edge of the sleeve device 106 by a release joint 120 or by electrolytic action or a dissolvable connection there-between. The diameter of the main body portion 122 of the mesh sleeve 106 is such as to permit blood flow “B” around the circumference thereof, as well as blood flow through the openings 124 within the mesh. This inverted form 116 of the tapered sleeve thus permits a mesh material of fairly relatively wider openings 124 therethrough, because of the doubling back and folding over of the more manipulable material after its inversion so as to thus better inhibit embolitic material from escaping the aneurysm 115 once that device 106 has been inverted and left in place.

A further embodiment of the present invention comprises a distortable, flexible mesh-like implant 130, is shown in FIGS. 7A and 7B, having a rather bulbous distal or leading end 132, and a generally cylindrical body portion comprising its proximal end 134. The proximal end 134 is open to permit the use of a pushrod or control rod 136, allowing the expandable bulbous end 132 to be narrowed during its deployment within an aneurysm 138 between a pair of side branches 140 and 142, as represented in FIG. 7B. Upon release/removal of the control rod 136 from within the distal end 132 of the device 130, the bulbous distal portion 132 assumes its final, enlarged configuration with a slightly tapering proximal portion 140 of the bulbous end conformably nesting within the tapered orifice 142 defining the aneurysm 138. The tapered, narrowed-diameter portion of the body 134 of the present device 130 is foraminous and of sufficient narrow diameter, so as to permit blood flow “B” therearound and therethrough, while the bulbous portion 132 prevents embolitic material “S” from escaping into the side branches. Such a stent device 130 may be filled with an embolic material upon establishment within aneurysm 138.

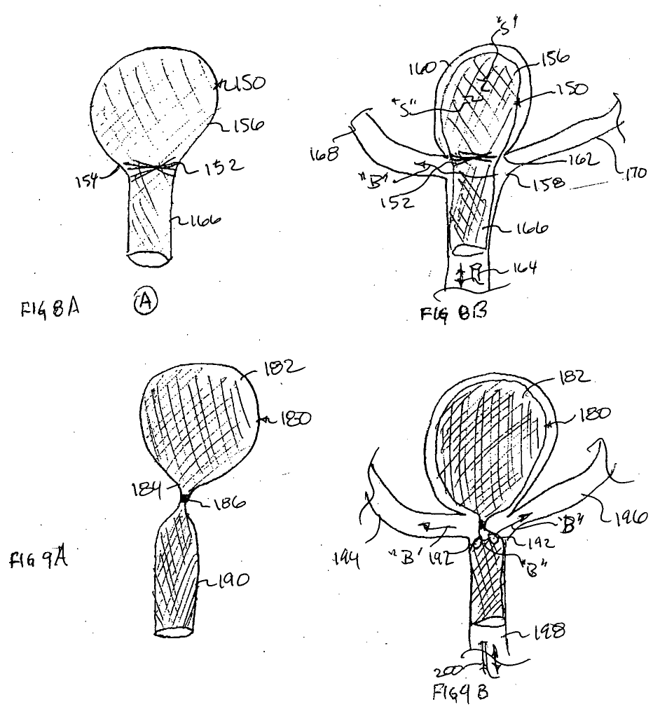

A bulbous device aneurysm. treatment device, 150, generally similar to the aforementioned embodiment, may include for example, a stage 152 or an arrangement of struts or shape memory members of metal or thermoplastic, as represented in FIGS. 8A and 8B, are pre-formed or inserted across the proximal portion 154 of the bulbous end 156 of the treatment device15O. The device 150 is positioned at a bifurcation 158, as represented in FIG. 6B, with its bulbous end 156 within an aneurysm 160. The staging, or cross strut arrangement 152, is positioned preferably within the orifice 162 of the aneurysm 160. A micro-catheter 164 may be positioned through or adjacent the proximal body portion 166 of the device to deliver embolic material into the aneurysm 160. The stage, or cross struts 152, which may for example, may be separately delivered via the micro-catheter 164, subsequent to placement of the device 150 within the aneurysm 160, help to contain the embolitic material “S” within the aneurysm 160, and prevent it from passing the transition point between the bulbous portion 156 and the tubular body portion 166 of the device 150. The embolitic material “S” will be utilized, of course, to fill the bulbous end 156 and support its treatment of the aneurysm 160. The struts or stage 152 are preferably in alignment with the orifice 162 of the aneurysm 160 to act as a partial wall between the branches 168 and 170 at the bifurcation.

A further embodiment of the bulbous cranial aneurysm treatment arrangement 180 over the earlier embodiments, is represented in FIGS. 9A and 9B, contemplated with a bulbous mesh distalmost end 182 having a generally mid-portion 184 pinched to a neck by a constriction band 186. The constriction band 186 separates the bulbous distal end 182 from the tubular end 190 of the implant device 180. The tubular end 190 of the implant deice 180 may be somewhat more porous, having larger openings therethrough or selectively having a plurality of machined or molded holes 192 positioned circumferentially about the tubular end 190, immediately adjacent the constriction band 186. Those large holes 192 would permit flow of blood “B” through, across, and between the various branches 194 and 196 subtending the parent vessel 198. The bulbous distal end 182 of the device 180 may be filled with a liquid or solid embolitic material, which has been delivered by a micro-delivery catheter 200 through the center of the tubular device 180 and the constriction band 186, or alongside the tubular portion 190 and directly into the distalmost bulbous portion 182 of the device 180. The holes 192, selectively positioned adjacent the constriction band 182, permits blood flow therethrough, as represented by the arrows “B” in FIG. 9B, as well as around the narrowed constricted portion 184 providing blood flow entirely around or through that segment of the device 180.

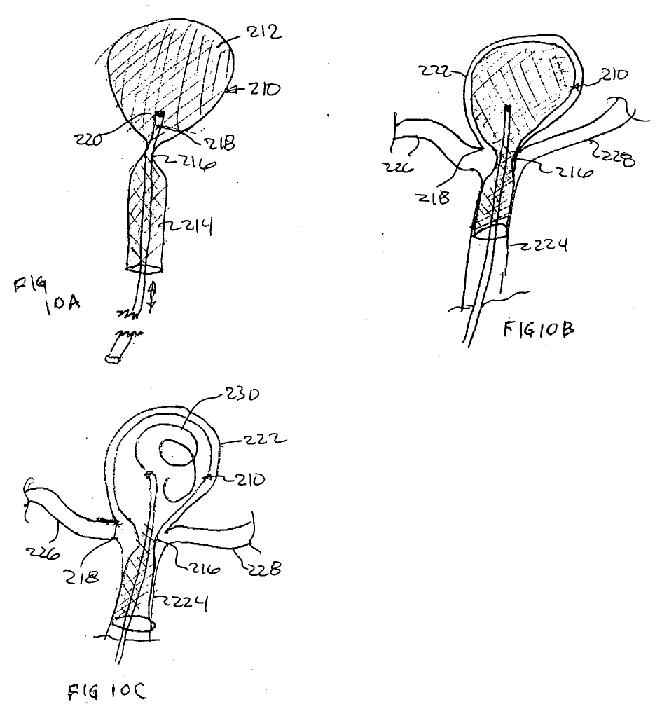

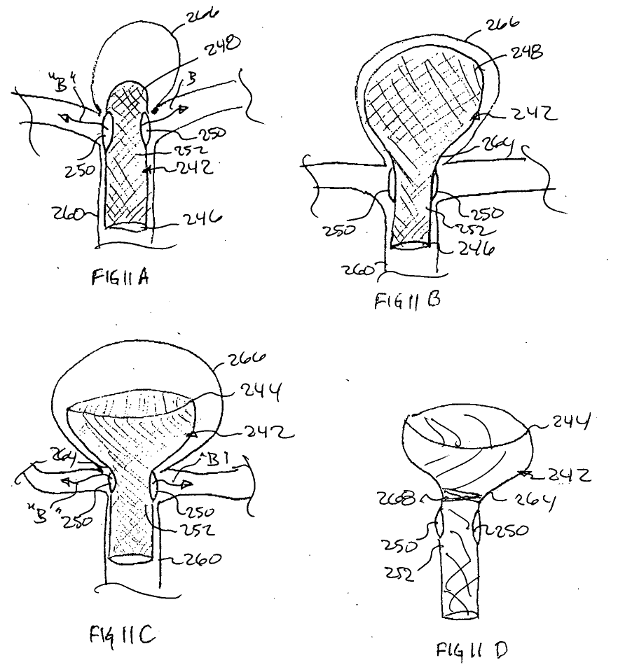

A further embodiment of the aneurysm treatment device 210, as represented in FIGS. 10A, 10B and 10C, comprises an elongated micro-catheter-delivered aneurysm treatment device 210 having a bulbous distal or leading end 212, and a tubular proximal, or trailing end 214, separated by a pinched waist portion 216 through which a micro-catheter delivery device 218 is pushed. The micro-catheter delivery device 218 has its distal end 220 within the bulbous portion 212 of the treatment device 210, with its pinched or transitioned waist portion 216 is best disposed just at the neck 218 of the aneurysm 222, as represented in FIGS. 10B and 10C, so as to minimize any potential blockage between the side branches 226 and 228 of the parent vessel 224. The micro-catheter delivery device 218, temporarily disposed within the bulbous portion of the treatment device 210, is utilized to deliver, for instance, an embolic coil 230 or other embolic material within the aneurysm 222 itself, as represented in FIG. 10C, the pinched or waist portion 216, minimizing any migration of embolic material from the aneurysm 222 itself.

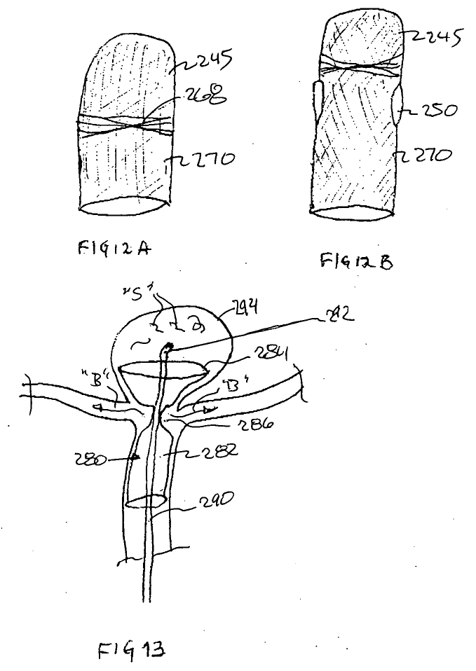

Further embodiments of the aneurysm treatment devices 240, are represented in FIGS. 11A, 11B, 11C and 11D, consist of mesh-like sleeves 242 having either open distalmost ends 244, as shown in FIGS. 11C and 11D, or bulbous closed distalmost ends 248 and open proximal ends 246, as represented for example, in FIGS. 11A and 11B. Each of these devices 242 is characterized with a plurality of side holes 250 arranged through the side wall portions 252 of the mesh device 242, so as to maximize flow of blood “B” between adjacent side branches of the parent vessel 260. The pinched, or tapered portions 264 of the treatment device 242 minimizes and/or restricts the embolitic material from moving out of the aneurysm 266 itself. Further embodiments of that same concept involve the use of struts or stages 268 immediately adjacent the side holes 250 within the wall 252 of the tubular portion of an elongated, more easily insertable, relatively straight walled treatment device 245, as represented for example, in FIGS. 12A and 12B, showing a tubular mesh device 270 with the insertable or pre-formed struts 268 and a mesh device 270 with the struts 268 and side holes 250 A still yet further embodiment of the present invention, shown in FIG. 13, includes an elongated aneurysm treatment device 280, having an open proximal body portion 282 and a cup-shaped distalmost portion 284, between which a pre-set pinch waist segment 286 is arranged. A micro-catheter 290 is extendable through the entire treatment device 280 having a distal end 292 extending beyond the distal end 284 of the treatment device 280, and centrally within the aneurysm 294 itself. Embolic material “S” is thus delivered within the aneurysm 294 and will be constrained therewithin. The pinched or waist portion 286, or alternatively a constriction band, is positioned at the bifurcation, so as to permit the flow of blood “B” there around.

Thus there has been shown a unique stent configurations and delivery arrangements for the treatment of intracranial aneurysms that occur at bifurcations of the brain such as, for example, the basilar apex, the middle cerebral artery, the anterior communicating artery, or the internal carotid bifurcation.

The invention thus comprises a stent for application within an aneurysm. The stent comprises an elongated tubular member having a proximal end and a distal end. The stent has its proximal portion expandable from a first diameter to a second diameter. The distal end of the stent is expandable to a third diameter. The third diameter is larger than the second diameter. The stent has an intermediate portion of a reduced diameter. The reduced diameter may comprise the first diameter. The intermediate portion may have an expansion restricting band element arranged thereon. At least one expandable strut may be arranged across the distal end of the stent. At least one expandable strut may be arranged within the stent and adjacent the distal end thereof. The distal end of the stent may be generally of a hemispherical shape when the distal end is expanded.

The invention may also include a method of treating an aneurysm at a bifurcation, comprising the steps of: introducing an elongated stent having a distal end and a proximal end through a parent vessel via a micro-catheter; inserting the distal end of the stent into the aneurysm; withdrawing the micro catheter from the stent; and expanding the distal end of the stent within the aneurysm. The method may also include one or more of the steps of: expanding a proximal portion of the stent within the parent vessel; maintaining a mid portion of the stent in an unexpanded configuration between a pair of branches of the bifurcation; expanding the distal portion of the stent to a greater diameter than the expanded diameter of the proximal portion; inserting at least one expandable strut across the distal end of the stent; and inserting at least one expandable strut within the distal end of the stent.

The invention may also include a method of treating an aneurysm at a bifurcation in the brain, comprising one or more of the following steps: introducing an elongated stent having a distal end and a proximal end through a parent vessel via a micro-catheter; inserting the distal end of the stent into the aneurysm and across a neck portion thereof; leaving the stent disposed within the aneurysm in which the stent is also left disposed across a neck portion of the aneurysm; and withdrawing the micro-catheter from the stent; leaving at least a proximal portion of the stent within the parent vessel unexpanded, while leaving the distal portion in an expanded configuration; and maintaining a mid portion of the stent in an unexpanded configuration between a pair of branches of the bifurcation. The invention in yet a further embodiment thereof, may include treatment of an aneurysm located anywhere in the body. The method may also include one or more of the following steps: inverting the distal portion of the stent so as to create a double walled portion thereof within the aneurysm being treated, arranging a plurality of holes within sidewalls of the stent to provide for maximum blood flow between adjacent branches of the bifurcation, arranging a plurality of struts across a portion of the stent to help define a wall at the neck of the aneurysm, arranging the struts within the stent prior to inserting the stent into an aneurysm, arranging the struts within the stent subsequent to inserting the stent into an aneurysm. The distal end of the stent may have a tapered tip thereon. The stent may be transformed from a tapered tip to a cup-shaped member after deployment within an aneurysm. The invention may also comprise a stent for treating a cranial aneurysm, including an elongated tubular member having a proximal end and a distal end, the stent having a proximal portion expandable from a first diameter to a second diameter, said distal end of the stent being expandable to a third diameter, the stent having a mid-portion with a pinched waist thereat. The proximal portion may have a plurality of enlarged holes therein, adjacent the pinched waist portion. The waist portion may have a constriction band therearound. The waist portion may have a plurality of enlarged holes thereadjacent. The distal end may comprise a bulbous member. The distal end may comprise a tapered tip. The waist portion is preferably arranged to permit a micro-catheter to pass therethrough.

The invention also includes a stent device for treating an aneurysm in a blood vessel, the stent comprising: an elongated tubular member having a proximal end and a distal end, the stent having a proximal portion expandable from a first diameter to a second diameter, the distal end of the stent being invertable by a control wire arranged through the stent and out the vessel. The distal end of the stent preferably has a tapered tip thereon, the tip being connected to the control wire. The stent device may have a marker band thereon to denote a neck portion of the stent device. The marker band may also comprise a neck constrictor. The invention also includes a method of treating an aneurysm in a body vessel, comprising the one or more of the following steps of: inserting a stent device with a control wire therewith, through a delivery catheter in the vessel, and into the aneurysm; inverting a distal portion of the stent device within the aneurysm by pulling proximally on the control wire; separating the control wire from the inverted distal tip of the stent device;

The invention also includes a method of establishing a generally hemispherically shaped aneurysm-treatment-device in an aneurysm within a body vessel, comprising one or more of the following steps; inserting an elongated stent device with a control wire therewith, through a delivery catheter in the vessel, and into the aneurysm; and inverting an aneurysm-occluding distal portion of the stent device within the aneurysm, so as to overlap the distal portion of the device onto a proximal portion of the stent device within the aneurysm; forming the aneurysm-occluding distal portion of the stent device from a self-inverting material to effect such inversion once the aneurysm-occluding portion of the stent device is within the aneurysm; manipulating the control wire proximally so as to effect inversion of the distal portion of the stent with the aneurysm.

We claim:

1. A stent for treating a cranial aneurysm, said stent comprising:
an elongated tubular member having a proximal end and a distal end, said stent having a proximal portion expandable from a first diameter to a second diameter, said distal end of said stent being expandable to a third diameter.

2. The stent as recited in claim 1, wherein said third diameter is larger than said second diameter.

3. The stent as recited in claim 1, wherein said stent has an intermediate portion of a reduced diameter.

4. The stent as recited in claim 3, wherein said reduced diameter comprises said first diameter.

5. The stent as recited in claim 3, wherein said intermediate portion has an expansion restricting band element arranged thereon.

6. The stent as recited in claim 1, including at least one expandable strut arranged across said distal end of said stent.

7. The stent as recited in claim 1, including at least one expandable strut arranged within said stent and adjacent said distal end thereof.

8. The stent as recited in claim 1, wherein said distal end of said stent is generally of a hemispherical shape when said distal end is expanded.

9. A method of treating an aneurysm at a bifurcation, comprising:
introducing an elongated stent having a distal end and a proximal end through a parent vessel via a micro-catheter;
inserting said distal end of said stent into said aneurysm and across a neck portion thereof;
withdrawing said micro-catheter from said stent; and
expanding said distal end of said stent within said aneurysm.

10. The method as recited in claim 9, including:
leaving at least a portion of said proximal portion of said stent unexpanded within said parent vessel.

11. The methods recited in claim 10, including:
maintaining a mid portion of said stent in an unexpanded configuration between a pair of branches of said bifurcation.

12. The method as recited in claim 11, including:
expanding said distal portion of said stent to a greater diameter than the expanded diameter of said proximal portion.

13. The method as recited in claim 9, including:
inserting at least one expandable strut across said distal end of said stent.

14. The method as recited in claim 9, including:
inserting at least one expandable strut within said distal end of said stent.

15. A method of treating an aneurysm at a bifurcation, comprising:
introducing an elongated stent having a distal end and a proximal end through a parent vessel via a micro-catheter;
inserting said distal end of said stent into said aneurysm and across a neck portion thereof;
leaving said stent disposed within said aneurysm in which said stent is also left disposed across a neck portion of said aneurysm;
withdrawing said micro catheter from said stent.

16. The method as recited in claim 15, including:
expanding at least a proximal portion of said stent within said parent vessel, while leaving said distal portion in an unexpanded configuration

17. The method as recited in claim 15, including:
maintaining a mid portion of said stent in an unexpanded configuration between a pair of branches of said bifurcation.

18. The method as recited in claim 15, wherein said aneurysm is located anywhere in the body.

19. The method as recited in claim 15, including:
inverting said distal portion of said stent so as to create a double walled portion thereof within said aneurysm being treated.

20. The method as recited in claim 15, including:
arranging a plurality of holes within sidewalls of said stent to provide for maximum blood flow between adjacent branches of said bifurcation.

21. The method as recited in claim 15, including:
arranging a plurality of struts across a portion of said stent to help define a wall at the neck of said aneurysm.

22. The method as recited in claim 21, including:
arranging said struts within said stent prior to inserting said stent into an aneurysm.

23. The method as recited in claim 21, including:
arranging said struts within said stent subsequent to inserting said stent into an aneurysm.

24. The method as recited in claim 19, wherein said distal end of said stent has a tapered tip thereon.

25. The method as recited in claim 24, wherein said stent is transformed from a tapered tip to a cup-shaped member after deployment within an aneurysm.

26. A stent for treating a cranial aneurysm, said stent comprising:
an elongated tubular member having a proximal end and a distal end, said stent having a proximal portion expandable from a first diameter to a second diameter, said distal end of said stent being expandable to a third diameter, said stent having a mid-portion with a pinched=waist thereat.

27. The stent as recited in claim 26, wherein said proximal portion has a plurality of enlarged therein, adjacent said pinched waist portion.

28. The stent as recited in claim 26, wherein said waist portion has a constriction band therearound.

29. The stent as recited in claim 26, wherein said waist portion has a plurality of enlarged holes thereadjacent.

30. The stent as recited in claim 26, wherein said distal end comprises a bulbous member.

31. The stent as-recited in claim 26, wherein said distal end comprises a tapered tip.

32. The stent as recited in claim 26, wherein said waist portion is arranged to permit a micro-catheter to pass therethrough.

33. A stent device for treating an aneurysm in a blood vessel, said stent comprising:
an elongated tubular member having a proximal end and a distal end, said stent having a proximal portion expandable from a first diameter to a second diameter, said distal end of said stent being invertable by a control wire arranged through said stent and out said vessel.

34. The stent device as recited in claim 33, wherein said distal end of said stent has a tapered tip thereon, said tip being connected to said control wire.

35. The stent as recited in claim 33, wherein said stent device has a marker band thereon to denote a neck portion of said stent device.

36. The stent device as recited in claim 35, wherein said marker band comprises a neck constrictor.

37. A method of treating an aneurysm in a body vessel, comprising:
inserting a stent device with a control wire therewith, through a delivery catheter in said vessel, and into said aneurysm;
inverting a distal portion of said stent device within said aneurysm by pulling proximally on said control wire.

38. The method of treating as recited in claim 37, including:
separating said control wire from said inverted distal tip of said stent device.

39. A method of establishing a generally hemispherically shaped aneurysm-treatment-device in an aneurysm within a body vessel, comprising:
inserting an elongated stent device with a control wire therewith, through a delivery catheter in said vessel, and into said aneurysm; and
inverting an aneurysm-occluding distal portion of said stent device within said aneurysm, so as to overlap said distal portion of said device onto a proximal portion of said stent device within said aneurysm.

40. The method as recited in claim 39, including:
forming said aneurysm-occluding distal portion of said stent device from a self-inverting material to effect such inversion once said aneurysm-occluding portion of said stent device is within said aneurysm.

41. The method as recited in claim 39, including;
manipulating said control wire proximally so as to effect inversion of said distal portion of said stent with said aneurysm.
